(19) United States
(12) Patent Application Publication
(10) Pub. No.: US 2002/0026139 A1
(43) Pub. Date: Feb. 28, 2002

(54) Valve seat and valve

(76) Inventors: William Jeff Bertrand, Ventura, CA (US); Robert Cushing Hamlen, Edina, MN (US); Steve J. May, Minnetonka, MN (US); Mitchell Andrew Solis, Alpharetta, GA (US)

Correspondence Address: Curtis D. Kinghorn, Medtronic Inc., 7000 Central Avenue NE, Minneapolis, MN, 55432, US

(21) Appl. No.: 09/776,517

(22) Filed: Feb. 2, 2001

Related U.S. Application Data
(60) Provisional application No. 60/179,660, filed on Feb. 2, 2000.

(51) Int. Cl. A61M 5/00

(52) U.S. Cl. 604/9; 604/247

(57) ABSTRACT

A valve seat for a ball valve in an adjustable medical valve is described. The valve seat is formed as a cone in a manifold. The cone is formed by rotating a line that intersects a central axis of the valve seat around the central axis. The angle of the intersection between the line and the central axis is doubled to form the included angle of the valve seat. Generally, the narrower the included angle, the better the tolerance of the assembled valve for variations in the spring constant of the pressure regulating spring. Specifically, the present invention has included angles between 0 and 90 degrees with the preferred included angles being between 10 and 40 degrees. A valve embodying the valve seat is also disclosed.

### BACKGROUND OF THE INVENTION

1. Field of the Invention

This invention relates generally to surgically implanted physiological shunt systems and related flow control devices. More particularly, the present invention relates to a valve seat in a one-way flow control valve for controlling the flow of cerebrospinal fluid out of a brain ventricle and preventing backflow of fluid into the brain ventricle.

2. Description of Related Art

In the medical arts, to relieve undesirable accumulation of fluids it is frequently necessary to provide a means for draining a fluid from one part of the human body to another in a controlled manner. This is required, for example, in the treatment of hydrocephalus, an ailment usually afflicting infants or children in which fluids accumulate within the skull and exert extreme pressure and skull deforming forces.

In treating hydrocephalus, cerebrospinal fluid accumulated in the brain ventricles is typically drained away utilizing a drainage or shunt system including a catheter inserted into the ventricle through the skull, which is connected to a tube that conducts the fluid away from the brain to be reintroduced into the peritoneal cavity or into the vascular system, as by extending a distal catheter through the patient's jugular vein to the atrium portion of the heart.

To control the flow of cerebrospinal fluid and maintain the proper pressure in the brain ventricle, a pump or valve is placed in the conduit between the brain and the peritoneal cavity or the heart. An exemplary flow control device is found in U.S. Pat. No. 4,560,375.

Although such drainage systems have provided successful results, a problem of overdrainage of the cerebrospinal fluid from the brain ventricles sometimes exists. Overdrainage of cerebrospinal fluid may result in excessive reduction of the cerebrospinal fluid pressure within the brain ventricles and predispose the development of a subdural hematoma or hydroma, and excessive reduction of ventricular size leading to shunt obstruction because of impingement of the ventricular walls on the inlet holes of the ventricular catheter.

This overdrainage can be caused by the siphoning effect of hydrostatic pressure in the distal shunt catheter. The siphoning effect of hydrostatic pressure may be created by the elevation of the ventricular catheter inlet with respect to the distal catheter outlet (i.e., when the patient sits, stands or is held erect). In order to prevent such overdrainage caused by the siphoning effect of hydrostatic pressure in the distal shunt catheter, siphon control devices have been placed in the conduit, typically between the flow control device and the peritoneal cavity or the heart. An exemplary siphon control device is found in U.S. Pat. No. 4,795,437.

It is desirable in some instances to permit the physician to be able to alter the flow characteristics through the drainage system after it has been subcutaneously implanted. To this end, on-off devices have been provided for implantation as a portion of the fluid conduit as an additional element of the shunt. An exemplary on-off device is shown in U.S. Pat. No. 3,827,439. Moreover, flow control devices have been provided which utilize a plurality of flow control valves having different flow control characteristics, which provide, alternative fluid pathways therethrough such that selection of a desired fluid pathway can be made by the selective percutaneous manipulation of the device when subcutaneously implanted. Such flow control devices having selectable alternative fluid pathways are shown in U.S. Pat. Nos. 5,154,693 and 5,167,615, the contents of which are incorporated herein.

These prior fluid shunt devices have all shared one important limitation: they only permit fluid flow therethrough upon achieving at most two fluid pressure differentials at the inlet and outlet of the device. In treating hydrocephalus, however, it is often desirable to vary the device “opening” pressure differential in accordance with ventricle size and treatment objective, For example, initial treatment may require a lower than normal pressure differential to initiate shrinkage of the ventricles, but as the ventricles decrease in size, the pressure differential should be increased gradually so that when the ventricle is returned to normal size the intraventricular pressure is at its normal value and the intracranial force systems are in balance (i.e., the opening differential pressure is set at a level that will stabilize the ventricles at a desired size). Generally speaking, the opening differential pressure should be varied inversely with the ventricle size. It is desirable to leave a lower pressure valve in a patient after the ventricles are again normal size, because the ventricles can further collapse, leading to a condition known as “slit” ventricles.

A further reason for providing adjustability in the opening pressure differential is to correct for variations in nominal opening pressure differentials typical in manufactured valves. With an adjustable valve, the opening pressure differential can be more accurately set at the factory and can be checked and corrected if necessary in the operating room prior to implantation.

Accordingly, there was a need in the medical arts for convenient and effective physiological drainage systems for controlling the flow of fluid from one part of the body to another, which are relatively inexpensive to manufacture, permit fluid flow therethrough only when upstream fluid pressure exceeds downstream fluid pressure by a selected pressure differential, and also provide means for altering the selected pressure differential by percutaneous manipulation of the device when it is subcutaneously implanted. Moreover, such a flow control device was needed that incorporates an integral siphon control device that opens only in response to positive upstream fluid pressure, and re-closes or remains closed in the absence of such positive upstream fluid pressure or in response to negative downstream hydrostatic pressure on the device.

These objectives were met in the invention described in U.S. Pat. No. 5,637,083, issued Jun. 10, 1997 to William J. Bertrand and David A. Watson entitled “IMPLANTABLE ADJUSTABLE FLUID FLOW CONTROL VALVE”, assigned to the assignee of the present invention, the contents of which are incorporated herein in its entirety. The invention described in the '083 patent resides in an improved subcutaneously implantable and percutaneously adjustable fluid flow control device useful in a physiological shunt system for controlling the flow of fluid from one part of the body to another. The fluid flow control device includes components responsive to an external or percutaneously-applied magnetic field, to provide the device a variety of pressure/flow characteristics.

In accordance with the invention described in the '083 patent, the fluid flow control device comprises an inlet, an outlet and valve means for controlling the fluid flow from the inlet to the outlet. The valve means comprises a valve housing including a fluid passageway therethrough which has a peripheral surface that forms a valve seat, and a valve element having a diameter larger than the valve seat. Means are provided for biasing the valve element against the valve seat so as to keep the fluid passageway closed until a fluid pressure differential between the inlet and the outlet exceeds a selected valve opening pressure. Further, a pump is situated between the inlet and the valve means. The pump provides means for flushing fluid through the device by the application of percutaneous pressure to the pump.

In one preferred form of the invention of the '083 patent, the valve housing includes a threaded aperture and a flow regulator insert which is threaded into the aperture to define the fluid passageway. Means are provided for adjusting the amount of bias applied to the valve element by the biasing means. In particular, the adjusting means includes a fixed dual concentric stair-step array and an overlying rotor assembly having a first surface which supports an end of a valve element-biasing spring, and a second surface which is supported by the stair-step array. The rotor assembly is adapted to rotate in response to an external or percutaneously-applied magnetic field and such rotation of the rotor assembly permits selected seating of the second surface on the stair-step array to raise or lower the rotor assembly with respect to the stair-step array.

The dual concentric stair-step array includes a central rotor pivot, a plurality of inner steps surrounding the rotor pivot, and a plurality of outer steps extending peripherally about the inner steps. The rotor assembly includes a magnet embedded within a base having an inner leg adapted to bear against a selected one of the plurality of inner steps, and outer leg disposed diametrically opposite the inner leg and adapted to bear against a selected one of the plurality of outer steps, a central aperture through which the rotor pivot extends, and a rotor cap fixed to the base on a side thereof opposite the inner and outer legs. The rotor cap provides the first surface of the rotor assembly and includes a central aperture aligned with the central aperture of the base, through which the rotor pivot extends.

A compression spring is provided between a portion of the valve housing surrounding the fluid passageway and the first surface of the rotor assembly. The compression spring biases the rotor assembly into contract with the dual concentric stair-step array.

Means are also provided for occluding a portion of the fluid flow control device adjacent to the inlet by application of manual percutaneous pressure to the device. Similarly, means are provided for occluding a portion of the fluid flow control device adjacent to the outlet also by application of manual percutaneous pressure to the device.

Moreover, a siphon control device is situated between the valve and the outlet.

In another preferred form of the invention of the '083 patent, means are provided for locking the rotor assembly into one of several possible rotational positions relative to the stair-step array to prevent rotation thereof. Further, means are provided for disengaging the locking means to permit rotation of the rotor assembly in response to the external magnetic field. More particularly, the locking means comprises a pin having a first end that engages one of a plurality of detents in an outer peripheral surface of the rotor assembly to prevent rotation thereof.

The disengaging means comprises pin-actuating means for moving the pin between a first extended position, wherein the end of the pin engages one of the plurality of detents, and a second retracted position. The pin actuating means comprises a pivotable lever including a pin-engaging shaft that engages a second end of the pin, and a manually actuated lever disposed within the pump and biased so as to urge the pin into its first position.

A disadvantage of the invention described in the '083 patent is that the cone angle of the valve seat assembly is very sensitive to the spring constant or rate of the pressure regulating spring.

### SUMMARY OF THE INVENTION

A valve seat for a ball valve in an adjustable medical valve is described. The valve seat is formed as a cone in a manifold. The cone is formed by rotating a line that intersects a central axis of the valve seat around the central axis. The angle of the intersection between the line and the central axis is doubled to form the included angle of the valve seat. Generally, the narrower the included angle, the better the tolerance of the assembled valve for variations in the spring constant of the pressure regulating spring. Specifically, the present invention has included angles between 0 and 90 degrees with the preferred included angles being between 10 and 40 degrees.

A valve embodying the valve seat and a method of using the valve is also disclosed.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a side cross-sectional view of the valve seat of the present invention in an adjustable flow control valve.

FIG. 2 is a close up side cross-sectional view of a portion of the valve seat of the present invention in an adjustable flow control valve.

FIG. 3 is a top view of the valve seat of the invention of FIG. 1.

FIG. 4 is a side cross-sectional view of the valve seat of the present invention with the ball seated in the valve seat.

FIG. 5 is a side cross-sectional view of a valve incorporating the valve seat of the present invention.

FIG. 6 is a side schematic view of a valve of the present invention in use in a hydrocephalus drainage system.

### DESCRIPTION OF THE INVENTION

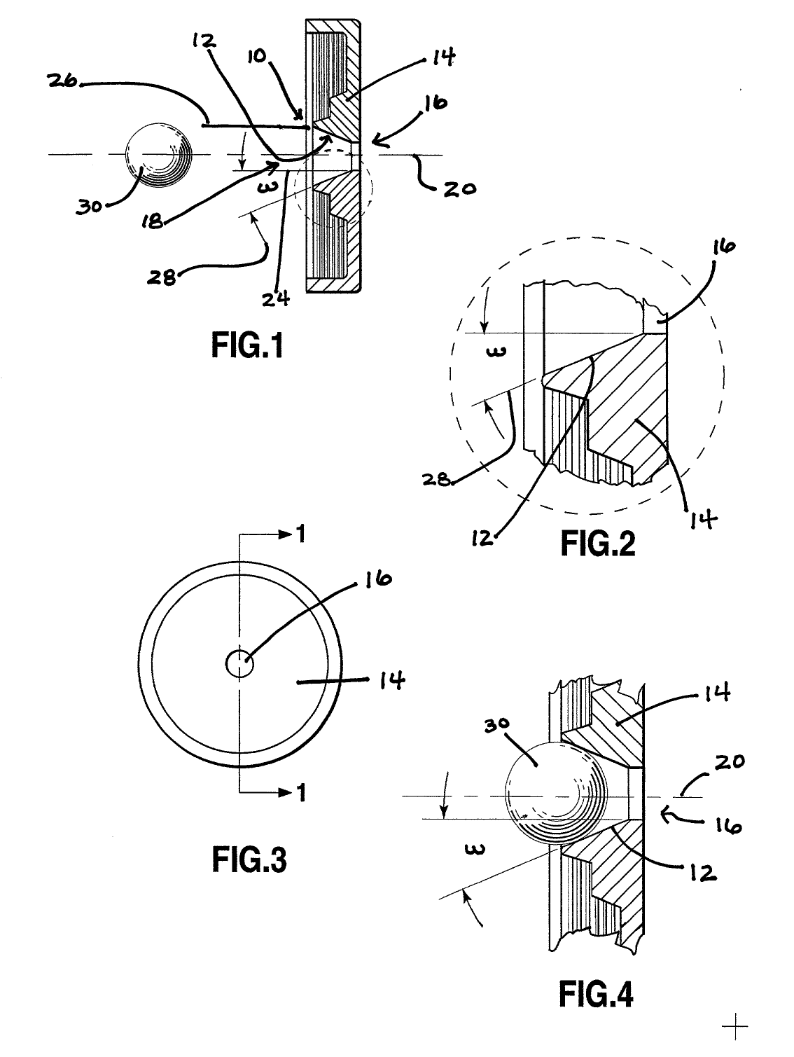

FIGS. 1-4 show a valve seat generally labeled 10. Valve seat 10 is defined by a conical wall 12 formed in a manifold 14 between a narrow circular opening 16 and a wide circular opening 18. A central axis 20 passes through the center of both the narrow circular opening 16 and the wide circular opening 18.

Narrow circular opening 16 is formed at the narrow end of valve seat 10 and wide circular opening 18 is formed at the wider end of valve seat 10. The radius of narrow circular opening 16 is equal to the distance from the central axis 20 to line 24 spaced from central axis 20. Wide circular opening 18 is formed at the wider end of valve seat 10. The radius of wide circular opening 18 is equal to the distance from the central axis 20 to a line 26 spaced from central axis 20.

As stated above, wall 12 is conical. Wall 12 is formed by rotating a line 28 that intersects central axis 20 around central axis 20. Line 28 intersects central axis 20 at an angle ω. The angle from one side of wall 12 to the other through central axis 20 is called “the included angle” and is equal to two times ω.

Ball 30 may be made of any rigid or semi-rigid material and is sized so that at least a portion of ball 30 contacts wall 12. In the preferred embodiment, ball 30 is made of ruby.

Computer hydrodynamic flow analysis has shown that the included angle of the valve seat 10 has a large effect on the performance of the valve. In general, this computer hydrodynamic flow analysis showed that the narrower the included angle of valve seat 10, the better the tolerance of the assembled valve for variations in the spring constant of the pressure regulating spring as is described in more detail below.

The preferred range of included angles for valve seat 10 is between 0 and about 90 degrees so that the preferred range of angles ω is between 0 and about 45 degrees. The most preferred included angle for valve seat 10 is between about 10 to about 40 degrees.

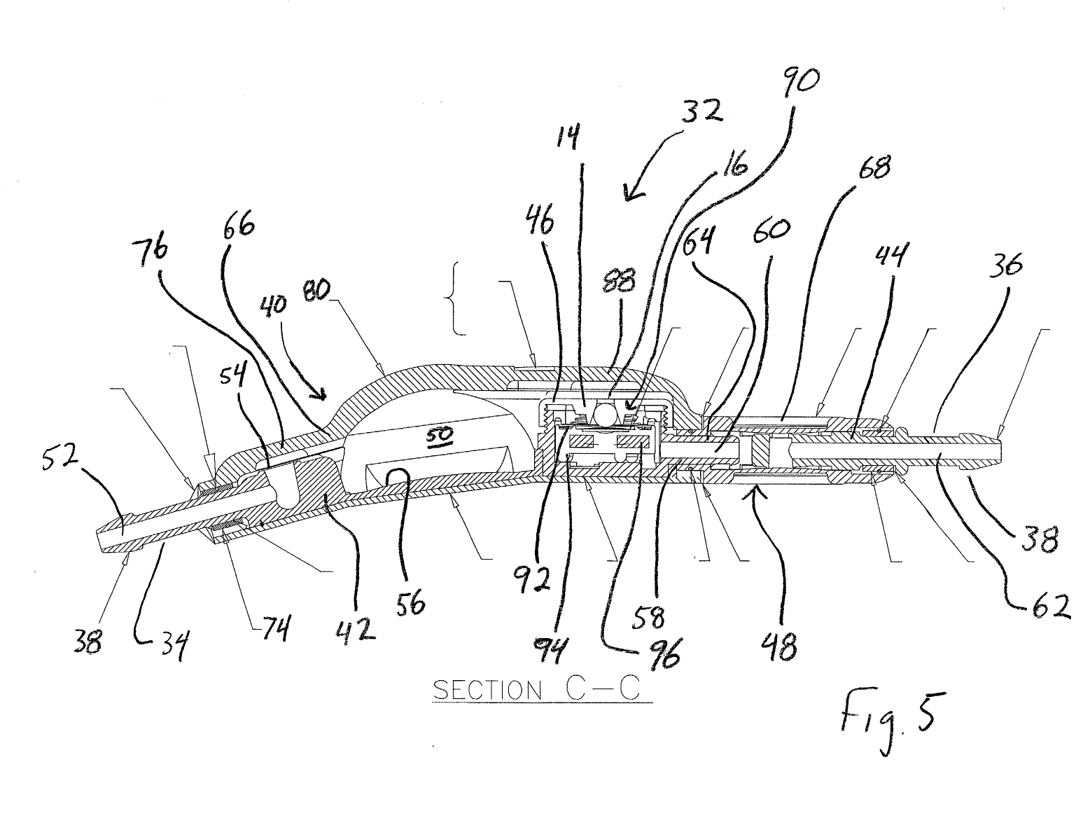

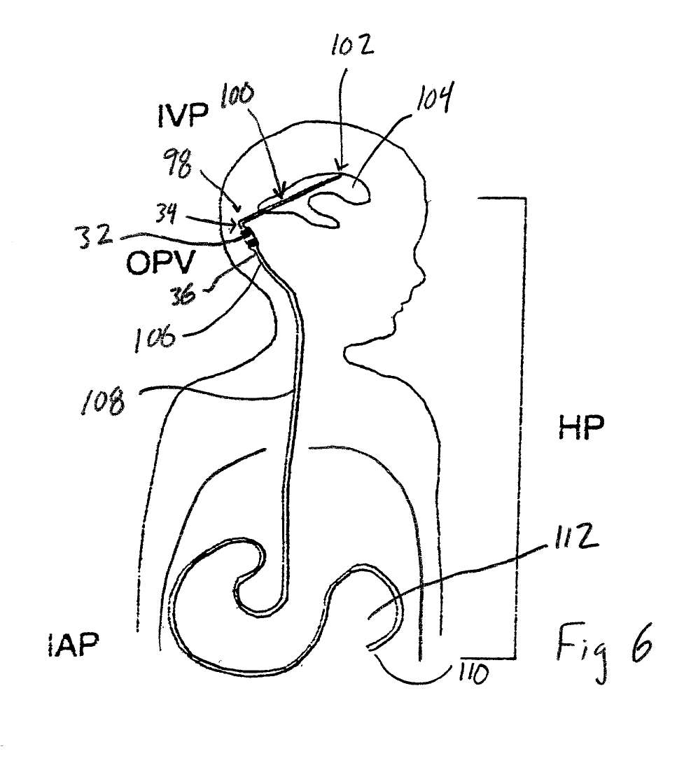

Valve seat 10 cooperates with a ball 30 as shown in FIG. 4 to form a valve 32 as shown in FIG. 5. Valve 32 includes an inlet connector 34 and an outlet connector 36. The inlet and outlet connectors 34, 36 will each be connected to catheters as will be described hereafter. The ends of the catheters are placed over the inlet and outlet connectors 34, 36 and preferably secured thereon by a single ligature just inside of an annular ridge 38 formed near the end of each connector.

In accordance with the present invention, the valve 32 includes a relatively rigid, molded plastic base invested within an elastomeric casing 40 which, together, define a fluid flow path through the valve 32 from the inlet connector 34 to the outlet connector 36. The base comprises an inlet section 42 integrally formed with the inlet connector 34, an outlet section 44 integrally formed with the outlet connector 36, and an intermediate valve housing 46 disposed within the elastomeric casing 40 between the inlet and outlet sections 34, 36. The valve housing 46 includes a percutaneously adjustable valve mechanism which restricts the flow of fluid through the valve 32 as is described in detail in U.S. Pat. No. 5,637,083, the teachings of which are incorporated herein in their entirety.

The casing 40 and the outlet section 44 of the base cooperate to provide a siphon control device 48 situated between the valve housing 46 and the outlet connector 36, which prevents fluid flow through the valve 32. The casing 40 and the inlet section 42 of the base cooperate to define a pump or flushing reservoir 50 between the inlet connector 34 and the valve housing 46.

The inlet section 42 defines an inlet flow channel 52 extending through the inlet connector 34 to an upwardly facing inlet occluder port 54. The inlet section 42 of the base forms a bottom plate 56 for the flushing reservoir 50 and an abutment support for a portion of the valve housing 46.

The valve housing 46 includes a snap-fit interlocking barbed connector 58. The barbed connector 58 extends from the valve housing 46 toward the outlet section 44 of the base, and forms a valve outlet fluid passageway 60 for directing fluids into the siphon control device 48. A pair of splines (not shown) extend from the valve housing 46 adjacent to the connector 58 and, together with the connector 58, interact with corresponding portions of the outlet section 44 of the base to prevent tensile and torsional movement of the valve housing 46 and the outlet section 44 of the base with respect to one another.

The outlet section 44 of the base is integrally formed with the outlet connector 36 which defines an outlet flow channel 62 therethrough. The outlet section 44 defines a portion of the siphon control device 48. A connector receptacle 64 is provided in the proximal end of the outlet section 44 for receiving the barbed connector 58 therein. Spline receiving slots (not shown) are provided in the proximal end of the outlet section 44 to sidably receive and substantially envelope the splines as the connector 58 is inserted into the receptacle 64. A similar base connection arrangement is illustrated in detail in U.S. Pat. No. 5,176,627, the contents of which are incorporated herein by reference in their entireties.

The elastomeric casing 40 is provided in two parts: a first or inlet casing body 66 into which the inlet section 42 of the base and the valve housing 46 are invested, and an outlet or second casing 68 which is sealed by a suitable adhesive to the inlet casing 66 in order to provide a continuous elastomeric exterior to the valve 32 with the exception of the inlet and outlet connectors 34 and 36 which extend therefrom. The inlet casing body 66 is integrally formed with the mounting pad 74 and includes an inlet aperture through which the inlet connector 34 extends, an inlet occluder wing 76 which generally overlies the inlet occluder port 54, and a resiliently flexible dome 80 for the flushing reservoir 50.

The inlet occluder wing 76 is positioned over the inlet occluder port 54 to facilitate occluding a portion of the fluid flow path through the valve 32 by pressing the wing 76 downwardly. Depressing the wing 76 and occluding the port 54 prevents proximal fluid flow from the flushing reservoir 50, defined by the dome 80 and the bottom plate 56, when the dome is pressed downwardly by manual percutaneous pressure,

The dome 80 is preferably molded of a silicone elastomer material and is designed to permit injection into the valve 32 by a hypodermic needle through the dome 80. The inlet section 42 of the base, as well as the outlet section 44 and the valve housing 46, is preferably molded of a polypropylene material which provides sufficient rigidity to prevent a needle from inadvertently passing through the valve 32 if an injection is made into the flushing reservoir 50. The construction of the base segments 42, 44 and 46, and the elastomeric casing 40, helps to guide a physician in manually percutaneously manipulating the valve 32 when subcutaneously implanted, for purposes of flushing the shunt system and adjusting the valve mechanism, when needed.

A distal occluder wing 88 is positioned over the valve housing 46 to facilitate occluding a narrow circular opening 16. This is accomplished by pressing the wing 88 downwardly, which effectively prevents distal fluid flow from the flushing reservoir 50 when the dome is pressed downwardly by manual percutaneous pressure.

The outlet casing body 68 surrounds a portion of the outlet section 44 of the base to define the siphon control device 48 which is similar to that shown and described in U.S. Pat. No. 4,795,437, the contents of which are incorporated herein by reference in their entirety.

With reference now to FIG. 5, the valve mechanism of the first illustrated embodiment of the valve 32 will be described in detail. The valve mechanism within the valve housing 46 includes manifold 14 with valve seat 10 formed therein as described above. The valve mechanism controls fluid flow from the inlet connector 34 to the outlet connector 36 and, more particularly, from the flushing reservoir 50 to the valve outlet fluid passageway 60. The valve housing 46 includes a flow regulator 90 described in detail in the '083 patent. The lower end of the flow regulator insert 90 forms the wide circular opening 18. Valve seat 10 is formed between narrow circular opening 16 and wide circular opening 18 as described above. Ball 30 seats against valve seat 10 to control the flow of fluid through the valve mechanism. Of course, to accomplish this the diameter of the ball 30 must be larger than the diameter of the valve seat 12 as described above.

A pressure spring 92 is disposed immediately below and in contact with the ball 30, to bias the ball 30 against the valve seat 12 until a fluid pressure differential between the inlet and the outlet exceeds a selected valve opening pressure.

The valve 32 of the present invention advantageously provides means for adjusting the amount of bias applied to the ball 30 by the pressure spring 116 in order to vary the selected valve opening pressure. Such adjusting means includes a fixed dual concentric stair-step array 94 and magnet 96 described in detail in the '083 patent above,

From the foregoing it is to be appreciated that the present invention provides a valve 32 for use in a subcutaneously implanted physiological shunt system, wherein the valve opening pressure may be selectively adjusted when subcutaneously implanted. The construction of the valve 32 of the present invention permits selective distal and proximal flushing of the devices through the application of manual percutaneous pressure. The present invention provides devices by which the flow of cerebrospinal fluid out of a brain ventricle can be controlled while preventing the backflow of fluid into the brain ventricle, and inhibiting excessive drainage through the physiological shunt in the presence of excessive downstream suction.

As stated above, the preferred range of included angles for the present invention begins at 0 degrees. Of course, the practical lower limit for the included angle is zero degrees. However, at zero degrees, the valve seat 10 will resemble a bore and the ball 30 will not seat in valve seat 10. That is, ball 30 will not rest against or contact wall 12 all around the periphery of ball 30 to form a seal. Instead, ball 30 will either pass through the valve seat 12 or not be able to enter it at all depending on the relative sizes of the valve seat bore at wide circular opening 18 and the ball 30.

For too narrow an included angle, such as those smaller than about 10°, the ball may get wedged or stuck in the valve seat 10. This problem is particularly true where the valve seat 10 is made of a polymer or a relatively compliant material and ball 30 is made of a more rigid material. But, such narrow included angles may be useful and an advantage when the valve seat 10 is made from a relatively rigid material such as a metal or ceramic and ball 30 is also made of a relatively rigid material.

In use, ball 30 will be biased in valve seat 10 so that ball 30 is biased toward narrow circular opening 16. In the preferred embodiment, ball 30 is biased toward the narrow circular opening 16 by a spring. Although a spring is the preferred embodiment to bias ball 30, other means to bias ball 30 include, but are not limited to hydraulic or pneumatic pressure, gravity, springs made or materials other than metal.

The spring characteristics of the spring, in combination with ball 30 and the size and included angle of valve seat 10, determine the flow characteristics of the valve. The use of a narrower included angle for valve seat 10 makes the valve assembly less sensitive to the spring constant or characteristics of the spring.

This is an extremely important manufacturing advantage over conventional methods of making valves where the pressure regulating springs are made from metal springs. In the conventional manufacturing methods, it is extremely difficult and requires extreme manufacturing precision to produce proper spring rates for the valve to work with the small pressures often found in hydrocephalous valves (0-20 cm H₂O). Modifying the angle of the valve seat 10 within the ranges presented herein is much easier to manufacture and allows a tolerance in the ranges of spring characteristics for the pressure regulating springs. This tolerance makes the manufacturing process for the valve more robust by easing the demands on spring manufacturing precision.

The valve, including the valve seat described above, may be used to treat hydrocephalus. This is done, as shown in FIG. 6, by fluidly connecting the proximal end 98 of a hydrocephalus catheter 100 to the inlet connector 34. An example of a hydrocephalus catheter 100 is a ventricular catheter such as model 41101 made and sold by Medtronic-PS Medical of Goleta, Calif. The distal end 102 of the hydrocephalus catheter 100 is placed in a patient's ventricle 104.

The distal end 106 of a drainage catheter 108 is fluidly connected to the outlet connector 36. An example of a drainage catheter 108 is a peritoneal catheter such as model 43522 made and sold by Medtronic-PS Medical of Goleta, Calif. The proximal end 110 of the drainage catheter 108 is placed in either a patient's venous system or a body cavity such as the peritoneal cavity 112.

The description contained herein is intended to be illustrative of the invention and not an exhaustive description. Many variations and alternatives to the disclosed embodiments will occur to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

1. A valve seat for a hydrocephalus valve, the valve seat having a central axis, the valve seat comprising:
a conical wall having a narrow circular opening and an opposed wide circular opening, the narrow circular opening and the wide circular opening being coaxial with the central axis, the conical wall being formed by rotating a line that intersects the central axis around the central axis, the line intersecting the central axis at an angle ω to form an included angle that is equal to two times ω.

2. The valve seat of claim 1 wherein the preferred range of included angles is between 0 and about 90 degrees.

3. The valve seat of claim 2 wherein the more preferred range of included angles is between about 10 to about 40 degrees.

4. A hydrocephalus valve comprising:
a valve seat, the valve seat having a central axis, the valve seat comprising a conical wall having a narrow circular opening and an opposed wide circular opening, the narrow circular opening and the wide circular opening being coaxial with the central axis, the conical wall being formed by rotating a line that intersects the central axis around the central axis, the line intersecting the central axis at an angle ω to form an included angle that is equal to two times ω;
a ball sized so that at least a portion of the ball contacts the wall around the periphery of the ball to form a seal.

5. The valve seat of claim 4 wherein the preferred range of included angles is between 0 and about 90 degrees.

6. The valve seat of claim 5 wherein the more preferred range of included angles is between about 10 to about 40 degrees.

7. The valve seat of claim 4 further comprising means for biasing the ball toward the narrow circular opening.

8. A method of treating hydrocephalus comprising the steps of:
providing a valve comprising:
a valve seat, the valve seat having a central axis, the valve seat comprising a conical wall having a narrow circular opening and an opposed wide circular opening, the narrow circular opening and the wide circular opening being coaxial with the central axis, the conical wall being formed by rotating a line that intersects the central axis around the central axis, the line intersecting the central axis at an angle ω to form an included angle that is equal to two times ω;
a ball sized so that at least a portion of the ball contacts the wall around the periphery of the ball to form a seal; and,
means for biasing the ball toward the narrow circular opening;
providing a hydrocephalus catheter having a distal and a proximal end;
connecting the proximal end of the hydrocephalus catheter to the narrow circular opening;
placing the distal end of the hydrocephalus catheter in a patient's ventricle;
providing a drainage catheter having a distal and a proximal end;
connecting the distal end of the drainage catheter to the wide circular opening;
placing the proximal end of the drainage catheter in either a patient's venous system, a body cavity or connected to an external drainage system.
